(19) United States
(12) Patent Application Publication
(10) Pub. No.: US 2023/0181267 A1
(43) Pub. Date: Jun. 15, 2023

(54) SYSTEM AND METHOD FOR INSTRUMENT EXCHANGE IN ROBOTIC SURGERY TRAINING SIMULATORS

(71) Applicant: Covidien LP, Mansfield, MA (US)

(72) Inventors: Max L. Balter, Boston, MA (US); Michael A. Eiden, Somerville, MA (US); Leslie E. Johnston, Hyde Park, MA (US); Tuvia C. Rappaport, Somerville, MA (US)

(21) Appl. No.: 18/080,050

(22) Filed: Dec. 13, 2022

Related U.S. Application Data
(60) Provisional application No. 63/289,222, filed on Dec. 14, 2021.

(51) Int. Cl. A61B 34/00; A61B 34/30

(52) U.S. Cl. CPC A61B 34/25; A61B 34/30

(57) ABSTRACT

A surgical robotic system includes surgeon console having a user input device configured to generate a user input for controlling a simulated instrument, a primary display configured to display a graphical surgical simulation including a simulated instrument, and a secondary display configured to display a graphical user interface providing exchange of the simulated instrument. The system also includes a training simulation computing device operably coupled to the surgeon console having a master controller configured to receive input positions from the user input device and to output a drive command for the simulated instrument, and a simulation controller configured to simulate the simulated instrument.

### CROSS-REFERENCE TO RELATED APPLICATIONS

This application claims the benefit of the filing date of provisional U.S. Patent Application No. 63/289,222 filed on Dec. 14, 2021. The entire contents of which is incorporated by reference herein.

### BACKGROUND

Surgical robotic systems are currently being used in minimally invasive medical procedures. Some surgical robotic systems include a surgeon console controlling a surgical robotic arm and a surgical instrument having an end effector (e.g., forceps or grasping instrument) coupled to and actuated by the robotic arm.

Each of the components, e.g., surgeon console, robotic arm, etc., of the surgical robotic system may be embodied in a training simulator. Thus, when surgeons require practice with the surgical robotic systems, a simulator of the surgical robotic systems provides the surgeon with the ability to practice common techniques used in robotic surgical procedures.

### SUMMARY

This disclosure generally relates to a surgical robotic system including a training simulation computing device for providing surgeons with training exercises to practice robotic procedures by mapping input from a surgeon console to a virtual surgical robotic system.

According to one embodiment of the present disclosure, a surgical robotic system is disclosed. The surgical robotic system includes surgeon console having a user input device configured to generate a user input for controlling a simulated instrument, a primary display configured to display a graphical surgical simulation including a simulated instrument, and a secondary display configured to display a graphical user interface providing exchange of the simulated instrument. The system also includes a training simulation computing device operably coupled to the surgeon console having a master controller configured to receive input positions from the user input device and to output a drive command for the simulated instrument, and a simulation controller configured to simulate the simulated instrument.

Implementations of the above embodiment may include one or more of the following features. According to one aspect of the above embodiment, the graphical user interface may include a graphical representation of the simulated instrument. The graphical representation may also include a name of the simulated instrument. The secondary display may be a touchscreen. The graphical representation may be configured as a button. The graphical representation, when selected, is also configured to output a selection menu including a plurality of instruments. The simulation controller may be further configured to replace the simulated instrument in response to a selection of a different instrument from the plurality of instruments. The graphical surgical simulation may be configured to display exchange of the simulated instrument with the selected instrument.

### BRIEF DESCRIPTION OF DRAWINGS

FIG. 1 is a schematic illustration of a surgical robotic system including a control tower, a console, and one or more surgical robotic arms in accordance with aspects of the present disclosure;

FIG. 2 is a perspective view of a surgical robotic arm of the surgical robotic system of FIG. 1;

FIG. 3 is a perspective view of a setup arm with the surgical robotic arm of the surgical robotic system of FIG. 1;

FIG. 4 is a schematic diagram of a computer architecture of the surgical robotic system of FIG. 1 with a training simulator console coupled to a surgeon console;

FIG. 5 is a schematic diagram of the training simulator console of the computer architecture of the surgical robotic system of FIG. 1;

FIG. 6 is a first view of a simulated view of a surgical site displayed on a primary display of the surgeon console according to an embodiment of the present disclosure;

FIG. 7 is a first view of a graphical user interface displayed on a secondary display of the surgeon console according to an embodiment of the present disclosure; and

FIG. 8 is a second view of the graphical user interface displayed on the secondary display of the surgeon console according to an embodiment of the present disclosure.

### DETAILED DESCRIPTION

The presently disclosed surgical robotic systems are described in detail with reference to the drawings, in which like reference numerals designate identical or corresponding elements in each of the several views.

As will be described in detail below, the present disclosure is directed to a surgical robotic system which includes a surgeon console, a control tower, a training simulator console, and one or more mobile carts having a surgical robotic arm coupled to a setup arm. The training simulator console is configured to allow for practice of robotic procedures based on the selected training exercise in the training simulator console. The surgeon console receives user input through one or more interface devices, which, the training simulator console maps to a virtual surgical robotic arm. The virtual surgical robotic arm includes a controller, which is configured to process the movement command and to generate a simulated position command for virtually moving the virtual robotic arm in response to the movement command.

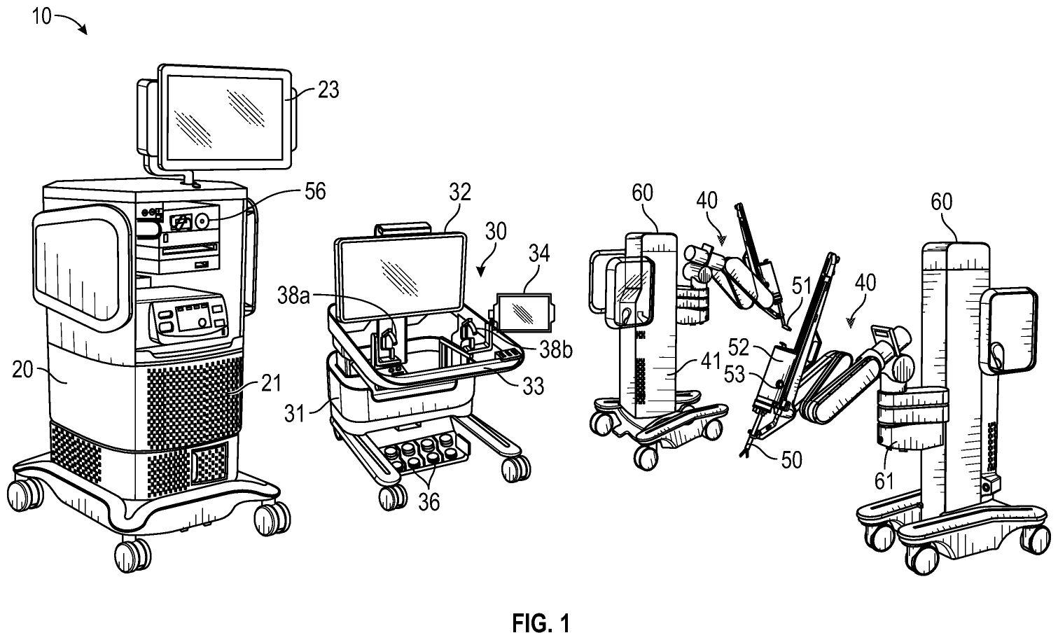

With reference to FIG. 1, a surgical robotic system 10 includes a control tower 20, which is connected to all of the components of the surgical robotic system 10 including a surgeon console 30 and one or more mobile carts 60. Each of the mobile carts 60 includes a robotic arm 40 having a surgical instrument 50 removably coupled thereto. The robotic arm 40 is also coupled to the mobile cart 60. The system 10 may include any number of mobile carts 60 and/or robotic arms 40.

The surgical instrument 50 is configured for use during minimally invasive surgical procedures. In embodiments, the surgical instrument 50 may be configured for open surgical procedures. In embodiments, the surgical instrument 50 may be an endoscope, such as an endoscopic camera 51, configured to provide a video feed for the user. In further embodiments, the surgical instrument 50 may be an electrosurgical forceps configured to seal tissue by compressing tissue between jaw members and applying electrosurgical current thereto. In yet further embodiments, the surgical instrument 50 may be a surgical stapler including a pair of jaws configured to grasp and clamp tissue while deploying a plurality of tissue fasteners, e.g., staples, and cutting stapled tissue.

One of the robotic arms 40 may include the endoscopic camera 51 configured to capture video of the surgical site. The endoscopic camera 51 may be a stereoscopic endoscope configured to capture two side-by-side (i.e., left and right) images of the surgical site to produce a video stream of the surgical scene. The endoscopic camera 51 is coupled to a video processing device 56, which may be disposed within the control tower 20. The video processing device 56 may be any computing device as described below configured to receive the video feed from the endoscopic camera 51 perform the image processing based on the depth estimating algorithms of the present disclosure and output the processed video stream.

The surgeon console 30 includes a first display 32, which displays a video feed of the surgical site provided by camera 51 of the surgical instrument 50 disposed on the robotic arm 40, and a second display 34, which displays a user interface for controlling the surgical robotic system 10. The first and second displays 32 and 34 are touchscreens allowing for displaying various graphical user inputs.

The surgeon console 30 also includes a plurality of user interface devices, such as foot pedals 36 and a pair of handle controllers 38a and 38b which are used by a user to remotely control robotic arms 40. The surgeon console further includes an armrest 33 used to support clinician's arms while operating the handle controllers 38a and 38b.

The control tower 20 includes a display 23, which may be a touchscreen, and outputs on the graphical user interfaces (GUIs). The control tower 20 also acts as an interface between the surgeon console 30 and one or more robotic arms 40. In particular, the control tower 20 is configured to control the robotic arms 40, such as to move the robotic arms 40 and the corresponding surgical instruments 50, based on a set of programmable instructions and/or input commands from the surgeon console 30, in such a way that robotic arms 40 and the surgical instruments 50 execute a desired movement sequence in response to input from the foot pedals 36 and the handle controllers 38a and 38b.

Each of the control tower 20, the surgeon console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area network, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 122.15.4-2003 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

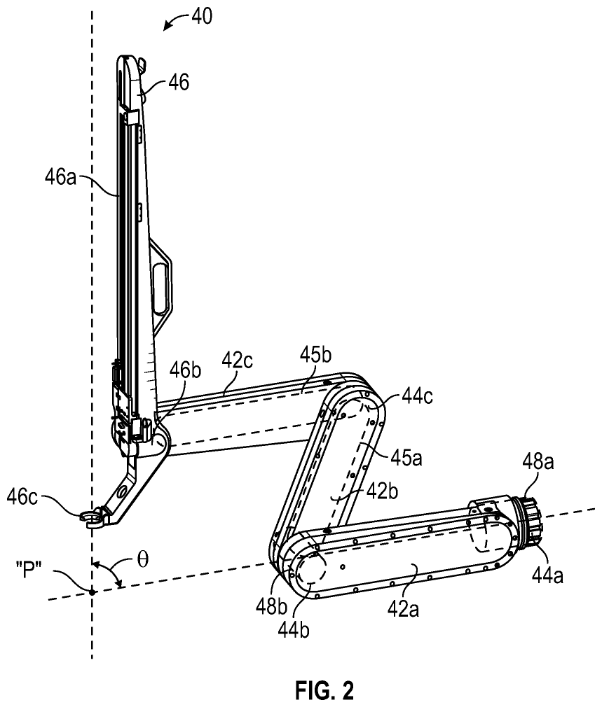

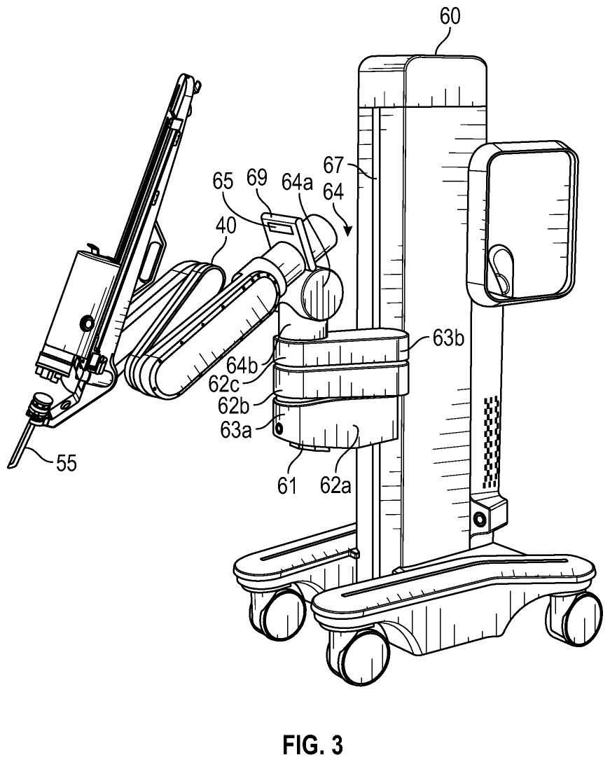

With reference to FIG. 2, each of the robotic arms 40 may include a plurality of links 42a, 42b, 42c, which are interconnected at joints 44a, 44b, 44c, respectively. Other configurations of links and joints may be utilized as known by those skilled in the art. The joint 44a is configured to secure the robotic arm 40 to the mobile cart 60 and defines a first longitudinal axis. With reference to FIG. 3, the mobile cart 60 includes a lift 67 and a setup arm 61, which provides a base for mounting of the robotic arm 40. The lift 67 allows for vertical movement of the setup arm 61. The mobile cart 60 also includes a display 69 for displaying information pertaining to the robotic arm 40. In embodiments, the robotic arm 40 may include any type and/or number of joints.

The setup arm 61 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In embodiments, the robotic arm 40 may be coupled to the surgical table (not shown). The setup arm 61 includes controls 65 for adjusting movement of the links 62a, 62b, 62c as well as the lift 67. In embodiments, the setup arm 61 may include any type and/or number of joints.

The third link 62c may include a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

The actuator 48b of the joint 44b is coupled to the joint 44c via the belt 45a, and the joint 44c is in turn coupled to the joint 46b via the belt 45b. Joint 44c may include a transfer case coupling the belts 45a and 45b, such that the actuator 48b is configured to rotate each of the links 42b, 42c and a holder 46 relative to each other. More specifically, links 42b, 42c, and the holder 46 are passively coupled to the actuator 48b which enforces rotation about a pivot point “P” which lies at an intersection of the first axis defined by the link 42a and the second axis defined by the holder 46. In other words, the pivot point “P” is a remote center of motion (RCM) for the robotic arm 40. Thus, the actuator 48b controls the angle θ between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle θ. In embodiments, some or all of the joints 44a, 44b, 44c may include an actuator to obviate the need for mechanical linkages.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

With reference to FIG. 2, the holder 46 defines a second longitudinal axis and configured to receive an instrument drive unit (IDU) 52 (FIG. 1). The IDU 52 is configured to couple to an actuation mechanism of the surgical instrument 50 and the camera 51 and is configured to move (e.g., rotate) and actuate the instrument 50 and/or the camera 51. IDU 52 transfers actuation forces from its actuators to the surgical instrument 50 to actuate components (e.g., end effector) of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c. During endoscopic procedures, the instrument 50 may be inserted through an endoscopic port 55 (FIG. 3) held by the holder 46. The holder 46 also includes a port latch 46c for securing the port 55 to the holder 46 (FIG. 2).

The robotic arm 40 also includes a plurality of manual override buttons 53 (FIG. 1) disposed on the IDU 52 and the setup arm 61, which may be used in a manual mode. The user may press one or more of the buttons 53 to move the component associated with the button 53.

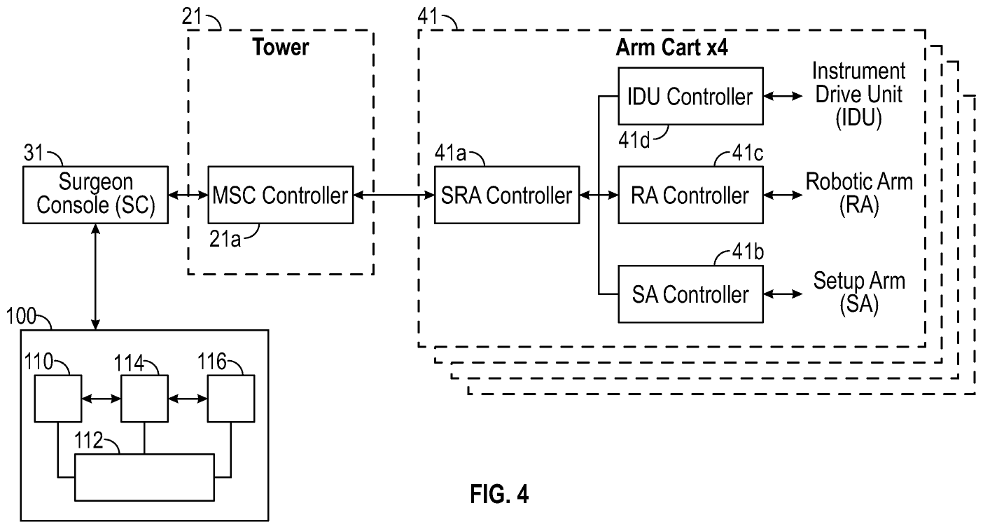

With reference to FIG. 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgeon console 30 about the current position and/or orientation of the handle controllers 38a and 38b and the state of the foot pedals 36 and other buttons. The controller 21a processes these input positions to determine desired drive commands for each joint of the robotic arm 40 and/or the instrument drive unit 52 and communicates these to the computer 41 of the robotic arm 40. The controller 21a also receives back the actual joint angles and uses this information to determine force feedback commands that are transmitted back to the computer 31 of the surgeon console 30 to provide haptic feedback through the handle controllers 38a and 38b. The safety observer 21b performs validity checks on the data going into and out of the controller 21a and notifies a system fault handler if errors in the data transmission are detected to place the computer 21 and/or the surgical robotic system 10 into a safe state.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the mobile cart 60, the robotic arm 40, and the instrument drive unit 52. The main cart controller 41a also communicates actual joint angles back to the controller 21a.

The setup arm controller 41b controls each of joints 63a and 63b, and the rotatable base 64 of the setup arm 62 and calculates desired motor movement commands (e.g., motor torque) for the pitch axis and controls the brakes. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the instrument drive unit 52. The IDU controller 41d calculates actual angles based on the motor positions and transmits the actual angles back to the main cart controller 41a.

The robotic arm 40 is controlled as follows. Initially, a pose of the handle controller controlling the robotic arm 40, e.g., the handle controller 38a, is transformed into a desired pose of the robotic arm 40 through a hand eye transform function executed by the controller 21a. The hand eye function, as well as other functions described herein, is/are embodied in software executable by the controller 21a or any other suitable controller described herein. The pose of handle controller 38a may be embodied as a coordinate position and role-pitch-yaw (“RPY”) orientation relative to a coordinate reference frame, which is fixed to the surgeon console 30. The desired pose of the instrument 50 is relative to a fixed frame on the robotic arm 40. The pose of the handle controller 38a is then scaled by a scaling function executed by the controller 21a. In some instances, the coordinate position may be scaled down and the orientation may be scaled up by the scaling function. In addition, the controller 21a also executes a clutching function, which disengages the handle controller 38a from the robotic arm 40. In particular, the main cart controller 21a stops transmitting movement commands from the handle controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the handle controller 38a and is then passed by an inverse kinematics function executed by the controller 21a. The inverse kinematics function calculates angles for the joints 44a, 44b, and 44c of the robotic arm 40 that achieve the scaled and adjusted pose input by the handle controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

With continued reference to FIGS. 1 and 4, the surgeon console 30 further includes a training simulation computing device 100 operably coupled to the surgeon console 30. The training simulation computing device 100 is configured to simulate operation of the surgical robotic system 10 (e.g., clutching, camera control, suturing, and stapling) based on a set of programmable instructions and/or input commands from the surgeon console 30 via the handle controllers 38a and 38b and the foot pedals 36. The training simulation computing device 100 simulates, in response to programmable instructions and/or input commands, virtual instances of the control tower 20, one or more mobile carts 60, the robotic arm 40, the surgical instrument 50, and the camera 51 disposed along with the surgical instrument 50 on the robotic arm 40.

The training simulation computing device 100 may include one or more computers, each including a plurality of controllers, namely, a master controller 110, a simulation controller 114, and a simulator 116 operably connected to a shared memory 112. The master controller 110 simulates the controller 21a. The shared memory 112 is configured to store session data and instrument information. The session data contains information such as, a scenario name, an initial position of an instrument, name of the instrument, and functionality of the instrument, e.g., whether instruments operate with electrosurgical generators, staple tissue, etc. The initial position of the instrument includes the pivot point “P” e.g., a tool center point (TCP) and joint 46b of holder 46, e.g., the remote center of motion (RCM). Optionally, the name of the instrument may be encoded in a vector look-up table, e.g., 256×1 vector, identified by a number corresponding to an instrument identifier including additional instrument information and may be received from the simulator 116.

The instrument information may include a maximum joint limit, a minimum joint limit of the surgical instrument 50, kinematic parameters of the instrument 50 (e.g., jaw offset and wrist length), an actual position of the surgical instrument 50 and camera 51, jaw opening ratios, and active instrument functions. The shared memory 112 may further include additional information, such as, state of the main cart controller 41a, active energy states, and initial exercise information.

Master controller 110 and the simulation controller 114 may be implemented in a computer, which may be running a Unix or Linux operating system, e.g., QNX, and the simulator 116 may be implemented in another computer, which may be running WINDOWS® operating system. The master controller 110 and the simulator 116 may be interconnected using any suitable communication network based on wired or wireless communication protocols. It should be understood that each of the master controller 110, the simulation controller 114, and the simulator 116 may be implemented in any combination of computers, interconnected to the one or more computers using any suitable communication network based on wired or wireless communication protocols. In some instances, the master controller 110 and the simulation controller 114 may be interconnected through one or more transmission protocols, including machine-to-machine communication protocols, such as a Data Distribution Service protocol for Real-Time Systems (DDS) including Real-Time Publish Subscribe Protocol (RTPS) enabling scalable, real-time, dependable, high performance, interoperable packet, or data exchanges. In some instances, the master controller 110 and the simulator 116 may be setup as virtual machines.

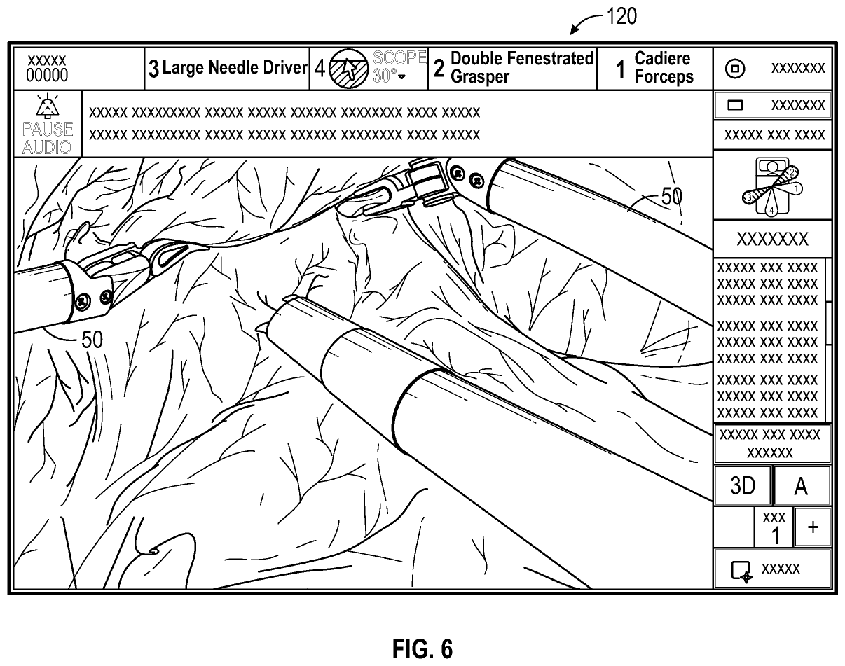

The simulator 116 of the training simulation computing device 100 simulates the commands and responses of the computer 41 including the main cart controller 41a, the setup arm controller 41b, the robotic arm controller 41c, and the instrument drive unit (IDU) controller 41d to and/or from the master controller 110. With reference to FIG. 6, the simulator 116 also outputs a simulated endoscopic view of the surgical site including simulated instruments 50 as well as their movements as imparted through the training simulation computing device 100. The endoscopic view is displayed a graphical simulation 120 on the first display 32 of the surgeon console 30.

The master controller 110 simulates the computer 21 of the control tower 20, including the controller 21a. In particular the master controller 110 receives session data from simulator 116 to determine desired drive commands for each joint, e.g., of the robotic arm 40 and/or the instrument drive unit 52, and communicates the desired drive commands and the instrument drive unit 52 to a virtual representation of the robotic arm 40 of the main cart controller 41a, which is simulated by the simulator 116 of the training simulation computing device 100.

The master controller 110 may be further configured to receive actual joint angles of the surgical instrument 50 to determine force feedback commands transmitted to the simulator 116 to provide haptic feedback through the handle controllers 38a and 38b of the surgeon console 30.

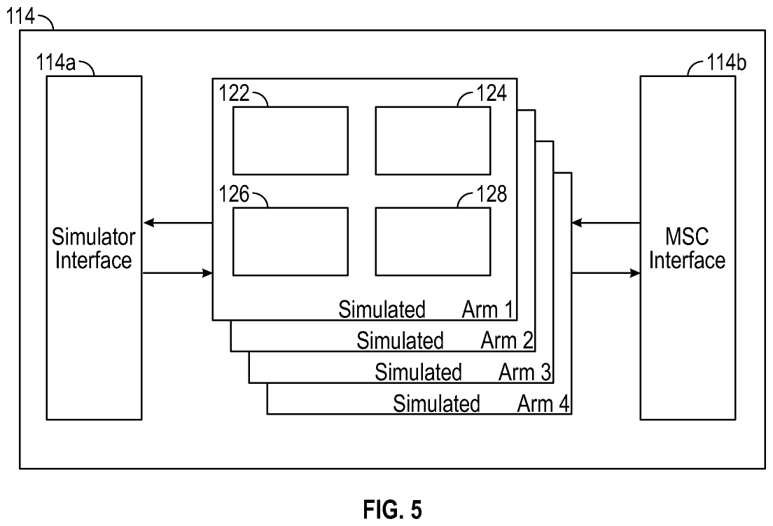

With reference to FIG. 5 the simulation controller 114 includes one or more communication interfaces. The communication interfaces include a simulator interface 114a and a simulation controller interface 114b. The simulator interface 114a is coupled to the simulator 116 and facilitates communication between the simulation controller 114 and the simulator 116. The simulation controller interface 114b is coupled to the master controller 110 and configured to facilitate communication between the master controller 110 and the simulation controller 114. The simulation controller 114 further includes an exercise initializer unit 122, a kinematics algorithm unit 124, a machine state unit 126, and an instrument function handler 128 for each robotic arm 40 simulated in the training simulation computing device 100. As used herein below, the robotic arm 40 and the associated components, e.g., joints 44a, 44b, 44c, instrument 50, etc. are referenced by the same numerals as the physical counterparts of FIG. 4 for simplicity, however, they are simulated by the simulation controller 114.

The machine state unit 126, based on commands received from the master controller 110, is configured to determine the appropriate action in the simulator 116 corresponding with a machine state. The machine state unit 126 may include one or more states, such as a registration state, a tele-robotic operation control state, and instrument specific states, e.g., a clip applier state, an electrosurgical state, and a stapler state. The registration state includes an “unregistered” and “registered” state. The registration state is initially set to a default state of “unregistered,” when the session is not active, and the simulated mobile cart is placed in a bedside active state to prevent tele-robotic operation control. When the session is active, the registration state is changed from “unregistered” to “registered” to allow tele-robotic operation control. The instrument specific states, may include: “disabled,” “wait clip reload,” and “reload animation” for a clip applier; “disabled,” “enabled,” “idle,” and “cutting” for electrosurgical forceps; and “disabled,” “idle,” “advancing,” “advancing paused,” and “advancing complete” for a stapler.

The tele-robotic operation control state includes a “waiting” and “ready” state. The “ready” state may further include sub-states, such as “hold,” “teleoperable,” and instrument specific states. The tele-robotic operation control state is initially set to a default state of “waiting” until the session is active. When the session is active, the tele-robotic operation control state is changed from “waiting” to “ready,” indicating to the master controller 110 that the mobile cart is ready for tele-robotic operation with a sub-state of “hold” until the mobile cart receives a command from the master controller 110 to enter tele-robotic operation. When tele-robotic operation is entered, the sub-state is changed from “hold” to “teleoperable” state. The sub-state may be changed back and forth from “hold” to “teleoperable,” based on a command received from the master controller 110. If the instrument 50 is a stapler and in the process of being reloaded, the sub-state may be changed from “teleoperable” to “reload animation” to disable tele-robotic operation during the reload animation.

The instrument function handler 128 maps instrument specific commands from the master controller 110 and the states from the machine state unit 126 to corresponding instrument functions within the training simulation computing device 100. The state of instrument 50 is received from the machine state unit 126. Based on the received state of instrument 50 and the specific command from the master controller 110, the command from the master controller 110 is mapped to the appropriate corresponding simulated instrument 50. The kinematics algorithm unit 124 is configured to perform kinematic calculations, such as inverse and forward kinematic calculations.

The exercise initializer unit 122 is configured to obtain the stored session data and instrument information from the simulator 116 to calculate an orientation and joint positions of joints 44a, 44b, and 44c of the simulated robotic arm 40 in a virtual fixed frame. The virtual fixed frame is a virtual representation of the fixed frame on the robotic arm 40, including one or more subset frames, such as, a TCP frame and an RCM frame. In some systems, the active instrument functions may be determined based on applying bit-masking to the incoming data corresponding to various functionality of the instruments, e.g., electrosurgical generators, staple tissue, etc.

To calculate the orientation of robotic arm 40, the initial instrument information, including an initial position of the instrument 50 and camera 51 is determined based on the initial TCP position relative to the RCM position. Instrument distances are calculated based on the difference between the initial TCP position and the RCM position (RCM-TCP). Based on the calculated instrument distances, x-direction (RCM-TCPₓ), y-direction (RCM-TCP_{y}), and z-direction (RCM-TCP_{z}) are calculated. Thus, the x-direction, y-direction, the z-direction, and the initial TCP position are combined to create an initial instrument pose (e.g., position and orientation). The initial instrument pose is post-multiplied by a transformation matrix to compensate for the hand eye coordination implemented in the master controller 110, resulting in an initial position of camera 51.

To calculate the initial joint positions of joints 44a, 44b, 44c of the simulated robotic arms 40, the kinematic algorithm unit 124 calculates a subset of the joints of the simulated robotic arms 40 (e.g., joints 44a, 44b, and 44c) from the RCM-TCP distances while the remaining joints are set to zero (0). The calculated subset of the joints 44a, 44b, and 44c of the robotic arms 40 is further processed through the kinematic algorithm unit 124 to calculate the TCP in the RCM frame for each instrument 50 and camera 51. The inverse of the calculated TCP in the RCM frame provides the RCM in the TCP frame. To determine the orientation of each simulated robotic arm 40 based in the virtual fixed frame, the RCM in the TCP frame is post-multiplied by initial pose of instrument 50 and camera 51, the results may be used in the master controller 110 to calculate the hand eye coordination, as well as further calculation in the kinematic algorithm unit 124.

The kinematic algorithm unit 124 is further configured to calculate desired simulated instrument poses from desired joint positions of the robotic arm 40 and an actual joint positions of the robotic arm 40 from actual poses of simulated instrument 50. The desired joint position of the robotic arm 40 is obtained from a position of the handle controllers 38a and 38b and/or foot pedals 36. The position of the handle controllers 38a and 38b and/or foot pedals 36 may include coordinate position and RPY orientation to a coordinate in the surgeon console 30 relative to the robotic arm 40 in a virtual fixed frame. The kinematic algorithm unit 124 calculates the desired positions of instrument 50 utilizing the desired joint positions of the robotic arm 40 from the master controller 110. The resulting desired poses of instrument 50 are post-multiplied with the RCM in the virtual fixed frame. In calculating the desired poses of camera 51, the desired poses of instrument 50 are further post-multiplied with the transpose of the calculated hand eye coordination in the master controller 110. In order to obtain the desired joint positions of the robotic arm 40, a switch having a time threshold may be implemented to ensure that the actual joint positions of the robotic arm 40 are initialized via the master controller 110 at the start of each exercise.

The kinematic algorithm unit 124 calculates the joint positions of the robotic arm 40 based on an average of the obtained actual positions of instrument 50 and the desired positions of instrument 50 post-multiplied with the inverse of the RCM in the virtual fixed frame. The joint positions of the robotic arm 40 are further configured to be transmitted to the master controller 110 to determine force feedback.

The simulation controller 114 may further include timing configured to indicate the start and end of a session. In aspects, the simulation controller 114 may further include simulation controller writer configured to transmit the desired and actual joint positions based on the machine state. In the event, the simulation controller 114 is in a tele-robotic operable state, the actual joint positions of the robotic arm 40 are transmitted to the master controller 110 for force feedback calculations. Otherwise, the desired joint positions of the robotic arm 40 are transmitted to the master controller 110 to disable force feedback. In some systems, the simulation controller 114 further includes a GUI writer to transmit information (e.g., robotic arm status, camera head state, and registration confirmed status) to a GUI subsystem of the second display device 34a. The information displayed by the second display device 34a is displayed during an active session allowing input from the user. In some instances, the simulation controller 114 may further include a simulation controller reader configured to obtain the desired joint positions of the robotic arm 40 and commands from the master controller 110.

The simulation controller 114 may further include a simulator writer configured to transmit poses of instrument 50 and/or camera 51, jaw angles, and active instrument functions to the shared memory 112 for further calculation. The training simulation computing device 100 may further includes additional software components found in a physical surgical robotic system, such as logging and data management, process and deployment, graphical user interface, alarms and notifications, surgeon console software subsystem, and simulation control software subsystem software.

In operation, the training surgeon console 100 is coupled to the surgeon console 30. The user selects a training exercise in the training surgeon console 100. The simulator 116 of the training surgeon console 100 initializes a session. The start of the session may be flagged by the timing control feature of the simulation controller 114. The exercise initializer unit 122 initializes the session by calculating an initial instrument and camera positions based on the initial TCP and initial RCM positions. The simulator writer may transmit the initial instrument and camera positions to the simulator 116 to initialize the session. The session data and instrument information are read from the shared memory 112 by simulation controller 114. The simulation controller 114 calculates actual joint positions of the robotic arm 40 based on the actual positions of instrument 50 from the instrument information read from the shared memory 112 by simulation controller 114. The simulation controller writer may transmit and write the calculated actual joint positions of the robotic arm 40 to the master controller 110 for force feedback, in particular, in the event that a command is received from the master controller 110 indicating that the machine state of the simulation controller 114 is in a tele-robotic operable state. The master controller 110 receives desired joint positions of the robotic arm 40 and commands from the user input, and the simulation controller 114 calculates desired poses of instrument 50 and camera 51 based on the desired joint positions of the robotic arm 40 and commands. The simulation controller reader may obtain the desired joint positions of the robotic arm 40 and commands from the master controller 110. The simulation controller writer may transmit the desired joint positions of the robotic arm 40 calculated to the master controller 110 to disable force feedback, in particular, in the event that commands are received from the master controller 110 indicating that the machine state of the simulation controller 114 is in a tele-robotic non-operable state. The simulator 116 also displays the graphical simulation 120 including one or more instruments 50 on the first display 32 of the surgeon console 30 as shown in FIG. 6. The instrument function handler 128, based on the received commands from the master controller 110, maps the corresponding command with an instrument function within the simulator 116. To map the corresponding commands with the instrument function within the simulator 116, the simulation controller 114 determines which robotic arm 40 and instrument drive unit 52 to simulate, determines the machine state of the robotic arm 40, and instrument drive unit 52.

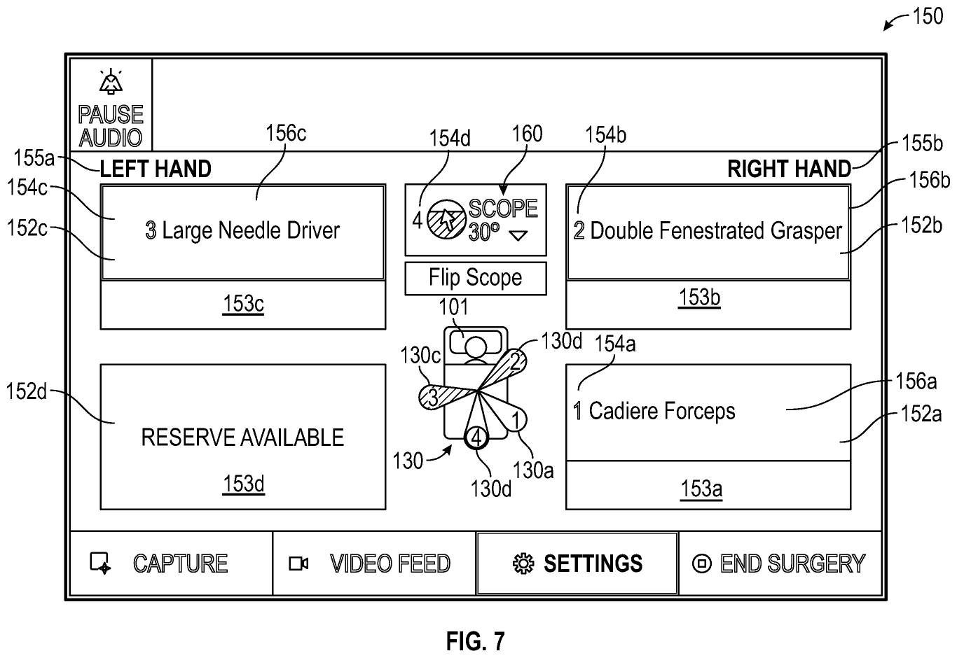

With reference to FIG. 7, a graphical user interface (GUI) 150 is displayed on the second display 34 of the surgeon console 30 and/or the display 23 of the control tower 20. The GUI 150 includes a plurality of regions 153a-d which include graphical representations 152a-c for each of the three robotic arms 40 numbered “1”-“3” and a reserve graphical representation 152d. Each of the graphical representations 152a-c includes an identification number 154a-c and an instrument type 156a-c. The GUI 150 also includes a region 160. The region 160 shows an arm identification number 154d and an orientation indicator for the indicator including pitch angle of the camera 51 and rotation relative to a horizontal plane. The region 160 also shows that the camera 51 is coupled to the robotic arm 40d numbered “4”. A fourth region 153d is reserved for reassigning any one of the graphical representations 152a-c. Similarly, the third region 153c may also be a placeholder.

The GUI 150 also shows a bed map 130 having a surgical table 101 and each of the robotic arms 40 represented as arrows 130a-d. The bed map 130 allows the users to quickly recognize the relationship of the corresponding mobile carts 60 to the surgical table 101. Each of the arrows 130a-d may display information pertaining to each of the corresponding mobile carts 60, such as an arm identification number, namely “1”-“4”, registered yaw angle, etc.

The mobile carts 60 may be automatically assigned to each of the graphical representations 152a-c, with the graphical representations 152a and 152b being controlled by the right-hand controller 38b and the graphical representations 152c and 152d being controlled by the left-hand controller 38a. However, the surgeon may move the instruments 50, i.e., robotic arms 40 between any of the four graphical representations 152a-d.

As noted above, the second display 34 is a touchscreen, which allows for moving the graphical representations 152a-d between the regions 153a-d by pressing, holding, and moving or using any other suitable touch gestures, e.g., moving the graphical representation 152a from the region 153a to any of the other regions 153b-d. This assigns the instrument to a desired one of the hand controllers 38a and 38b, designated as a left-hand column 155a and a right-hand column 155b, respectively. As the icons are moved between any of the graphical representations 152a-c, the user can confirm the actual physical location of the instruments 50 and their corresponding robotic arms 40a-d by matching the colors displayed on the GUI 150 to the colors on the color indicators 102a-d regardless of which graphical representation 152a-d is being used.

The master controller 110 automatically assigns the mobile carts 60 and corresponding instruments 50 to the regions 153a-c of the GUI 150. In embodiments, the master controller 110 may assign instrument mobile carts 60 in numerical order, based on the number, i.e., 1-3, of the mobile carts 60 such that the first arm cart 60a numbered “1” is assigned to the first region 153a, the second arm cart 60b numbered “2” is assigned to the second region 153b, and the third arm cart 60c numbered “3” is assigned to the third region 153c, with the fourth region 153d being held in reserve. However, occasionally instrument mobile carts 60 are positioned on one side (e.g., right) of the surgical table 101 but are automatically assigned to the opposite side handle controller 38a (e.g., left) due to the numbering of the instrument mobile carts 60. In embodiments, once the automatic assignment is completed the surgeon or the technician may manually move the graphical representation 152a-c to any of the regions 153a-d to correlate correct position of the mobile carts 60 to the regions 153a-d.

The master controller 110 is also configured to simulate exchange of instruments 50. During use of the surgical system 10, various instruments 50 may be used with corresponding robotic arms 40. In embodiments, a plurality of instruments 50 may be used with a single robotic arm 40 using an instrument exchange procedure, which includes extracting the instrument 50 from the patient, disconnecting the instrument 50 from the IDU 52, and connecting a new instrument to the IDU 52. During the instrument exchange, the IDU 52 is configured to communicate with the instrument 50 to identify the instrument 50 and update the surgical system 10 accordingly, e.g., update the GUI 150. However, during simulation, since no actual instruments 50 are used, the master controller 110 enables the GUI 150 to simulate instrument exchange.

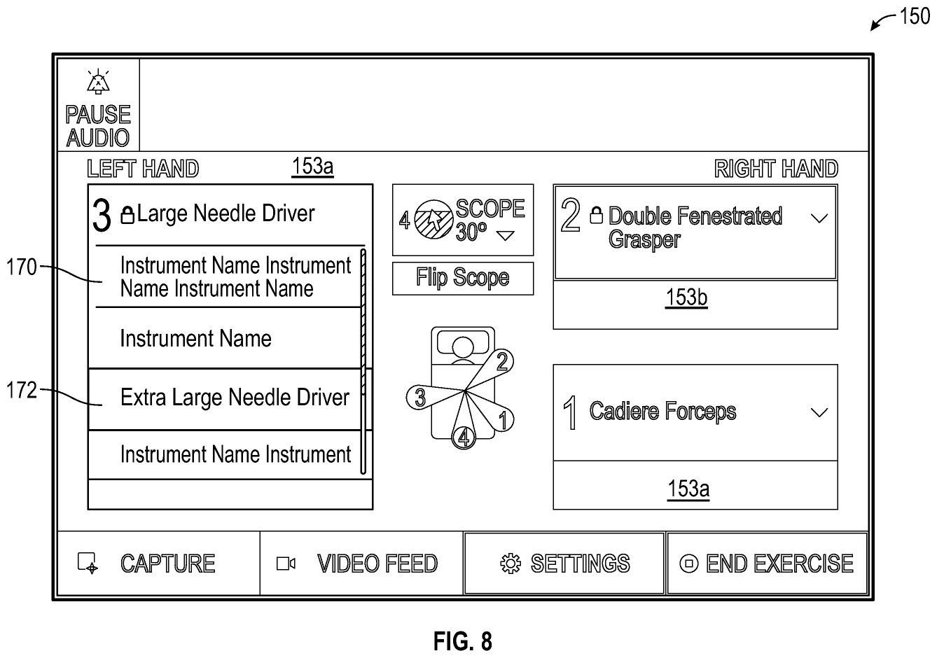

With reference to FIG. 8, to simulate instrument exchange, the user presses on one of regions 153a-c. In response to the press, the GUI 150 displays an instrument selection menu 170, which may be a drop-down menu, a grid, etc., displaying a plurality of instruments 50 that may be simulated by the master controller 110 on the graphical simulation 120. The user may then press on one of the selections 172 of the selection menu 170. In embodiments, eye-tracking hardware of the surgeon console 30 may be used to track surgeon's gaze, which may be used to open the instrument selection menu 170. Eye tracking may be used to scroll or otherwise navigate through the selections 172 and a confirmation of the instrument may be done by a pedal or button press. In further embodiments, voice commands may be used to opening the selection menu 170 and choosing a new instrument.

In response to the selection, an animation of the currently used instrument 50 being withdrawn is shown on the graphical simulation 120 and the selected instrument 50 is shown being inserted into the field of view of the graphical simulation 120. During the instrument exchange, the simulated robotic arm 40 also transitions to a manual mode, since instruments 50 are manually exchanged by surgical staff.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. A surgical robotic system comprising:
a surgeon console including:
a user input device configured to generate a user input for controlling a simulated instrument;
a primary display configured to display a graphical surgical simulation including a simulated instrument;
a secondary display configured to display a graphical user interface providing exchange of the simulated instrument; and
a training simulation computing device operably coupled to the surgeon console including:
a master controller configured to receive input positions from the user input device and to output a drive command for the simulated instrument; and
a simulation controller configured to simulate the simulated instrument.

2. The surgical robotic system according to claim 1, wherein the graphical user interface includes a graphical representation of the simulated instrument.

3. The surgical robotic system according to claim 2, wherein the graphical representation includes a name of the simulated instrument.

4. The surgical robotic system according to claim 2, wherein the secondary display is a touchscreen.

5. The surgical robotic system according to claim 4, wherein the graphical representation is configured as a button.

6. The surgical robotic system according to claim 4, wherein the graphical representation, when selected, is configured to outputs a selection menu including a plurality of instruments.

7. The surgical robotic system according to claim 6, wherein the simulation controller is configured to replace the simulated instrument in response to the selection of a different instrument from the plurality of instruments.

8. The surgical robotic system according to claim 7, wherein the graphical surgical simulation is configured to display exchange of the simulated instrument with the selected instrument.
